(19) United States
(12) Patent Application Publication
(10) Pub. No.: US 2005/0227999 A1
(43) Pub. Date: Oct. 13, 2005

(54) Diarylamine derivatives as calcium channel blockers

(75) Inventors: Hassan Pajouhesh, Vancouver (CA); Hossein Pajouhesh, Burnaby (CA); Yanbing Ding, Vancouver (CA); Terrance P. Snutch, Vancouver (CA)

Correspondence Address: MORRISON & FOERSTER LLP, 3811 VALLEY CENTRE DRIVE, SUITE 500, SAN DIEGO, CA, 92130-2332, US

(73) Assignee: NeuroMed Technologies Inc.

(21) Appl. No.: 10/928,564

(22) Filed: Aug. 27, 2004

Related U.S. Application Data
(63) Continuation-in-part of application No. 10/821,584, filed on Apr. 9, 2004.

(51) Int. Cl. A61K 31/495; C07D 241/04; C07D 43/02

(52) U.S. Cl. 514/255.04; 544/398; 544/360; 514/253.01

(57) ABSTRACT

Compounds which are derivatives of diarylamine substituted piperazine and amino-piperidine are useful in treating conditions mediated by calcium ion channel activity.

### CROSS-REFERENCE TO RELATED APPLICATIONS

This application is a continuation-in-part of U.S. Ser. No. 10/821,584, filed 9 Apr. 2004, which is incorporated herein by reference.

### TECHNICAL FIELD

The invention relates to compounds useful in treating conditions associated with calcium channel function. More specifically, the invention concerns compounds containing substituted or unsubstituted diarylamine derivatives of 6-membered heterocyclic moieties that are useful in treatment of conditions such as stroke and pain.

### BACKGROUND ART

The entry of calcium into cells through voltage-gated calcium channels mediates a wide variety of cellular and physiological responses, including excitation-contraction coupling, hormone secretion and gene expression (Miller, 1987; Augustine, et al., 1987). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter and calcium channels, which also affects neurite outgrowth and growth cone migration in developing neurons.

Calcium channels mediate a variety of normal physiological functions, and are also implicated in a number of human disorders. Examples of calcium-mediated human disorders include but are not limited to congenital migraine, cerebellar ataxia, angina, epilepsy, hypertension, ischemia, and some arrhythmias. The clinical treatment of some of these disorders has been aided by the development of therapeutic calcium channel antagonists (e.g., dihydropyridines, phenylalky amines, and benzothiazapines all target L-type calcium channels) (Janis and Triggle, 1991).

Native calcium channels have been classified by their electrophysiological and pharmacological properties into T-, L-, N-, P/Q- and R-types (reviewed in Catterall, 2000; Huguenard 1996). T-type (or low voltage-activated) channels describe a broad class of molecules that transiently activate at negative potentials and are highly sensitive to changes in resting potential.

The L-, N- and P/Q-type channels activate at more positive potentials (high voltage-activated) and display diverse kinetics and voltage-dependent properties (Catterall, 2000; Huguenard 1996). L-type channels can be distinguished by their sensitivity to several classes of small organic molecules used therapeutically, including dihydropyridines (DHPs), phenlyalkylamines and benzothiazepines. In contrast, N-type and P/Q-type channels are high affinity targets for certain peptide toxins produced by venous spiders and marine snails: N-type channels are blocked by the co-conopeptides ω-conotoxin GVIA (ω-CTx-GVIA) isolated from Conus geographus and ω-conotoxin MVIIA (ω-CTx-MVIIA) isolated from Conus magus, while P/Q-type channels are resistant to ω-CTx-MVIIA but are sensitive to the funnel web spider peptide, ω-agatoxin IVA (ω-Aga-IVA). R-type calcium channels are sensitive to block by the tarantula toxin, SNX-482.

Neuronal high voltage-activated calcium channels are composed of a large (>200 kDa) pore-forming α₁ subunit that is the target of identified pharmacological agents, a cytoplasmically localized ˜50-70 kDa β subunit that tightly binds the α₁ subunit and modulates channel biophysical properties, and an ˜170 kDa α₂δ subunit (reviewed by Stea, et al., 1994; Catterall, 2000). At the molecular level, nine different α₁ subunit genes expressed in the nervous system have been identified and shown to encode all of the major classes of native calcium currents (Table 1).

TABLE 1
Classification of Neuronal Calcium Channels

| | | | ω- | | | |
|---|---|---|---|---|---|---|
| Native | | Gene | AGA | ω-CTx | ω-CTx | |
| Class | cDNA | Name | IVA | GVIA | MVIA | dihydropyridines |
| P/ | α_{1A} | Caᵥ2.1 | ✓ | — | — | — |
| Q-type | | | | | | |
| N-type | α_{1B} | Caᵥ2.2 | — | ✓ | ✓ | — |
| L-type | α_{1C} | Caᵥ1.2 | — | — | — | ✓ |
| L-type | α_{1D} | Caᵥ1.3 | — | — | — | ✓ |
| R-type | α_{1E} | Caᵥ2.3 | — | — | — | — |
| L-type | α_{1F} | Caᵥ1.4 | — | — | — | ✓ |
| T-type | α_{1G} | Caᵥ3.1 | — | — | — | — |
| T-type | α_{1H} | Caᵥ3.2 | — | — | — | — |
| T-type | α_{1I} | Caᵥ3.3 | — | — | — | — |

Calcium channels have been shown to mediate the development and maintenance of the neuronal sensitization processes associated with neuropathic pain, and provide attractive targets for the development of analgesic drugs (reviewed in Vanegas and Schaible, 2000). All of the high-threshold Ca channel types are expressed in the spinal cord, and the contributions of L-, N and P/Q-types in acute nociception are currently being investigated. In contrast, examination of the functional roles of these channels in more chronic pain conditions strongly indicates a pathophysiological role for the N-type channel (reviewed in Vanegas & Schaible, 2000).

Mutations in calcium channel α₁ subunit genes in animals can provide important clues to potential therapeutic targets for pain intervention. Genetically altered mice null for the α_{1B} N-type calcium channel gene have been reported by several independent groups (Ino, et al., 2001; Kim, etal., 2001; Saegusa, etal., 2001; Hatakeyama, et al., 2001). The α_{1B} N-type null mice were viable, fertile and showed normal motor coordination. In one study, peripheral body temperature, blood pressure and heart rate in the N-type gene knock-out mice were all normal (Saegusa, et al., 2001). In another study, the baroreflex mediated by the sympathetic nervous system was reduced after bilateral carotid occlusion (Ino, et al., 2001). In another study, mice were examined for other behavioral changes and were found to be normal except for exhibiting significantly lower anxiety-related behaviors (Saegusa, et al., 2001), suggesting the N-type channel may be a potential target for mood disorders as well as pain. In all studies mice lacking functional N-type channels exhibit marked decreases in the chronic and inflammatory pain responses. In contrast, mice lacking N-type channels generally showed normal acute nociceptive responses.

Two examples of either FDA-approved or investigational drug that act on N-type channel are gabapentin and ziconotide. Gabapentin, 1-(aminomethyl) cyclohexaneacetic acid (Neurontin®), is an anticonvulsant originally found to be active in a number of animal seizure models (Taylor, et al., 1998). Subsequent work has demonstrated that gabapentin is also successful at preventing hyperalgesia in a number of different animal pain models, including chronic constriction injury (CCI), heat hyperalgesia, inflammation, diabetic neuropathy, static and dynamic mechanoallodynia associated with postoperative pain (Taylor, et al., 1998; Cesena & Calcutt, 1999; Field, et al., 1999; Cheng, J-K., et al., 2000; Nicholson, 2000).

While its mechanism of action is incompletely understood, current evidence suggests that gabapentin does not directly interact with GABA receptors in many neuronal systems, but rather modulates the activity of high threshold calcium channels. Gabapentin has been shown to bind to the calcium channel α₂δ ancillary subunit, although it remains to be determined whether this interaction accounts for its therapeutic effects in neuropathic pain.

In humans, gabapentin exhibits clinically effective anti-hyperalgesic activity against a wide ranging of neuropathic pain conditions. Numerous open label case studies and three large double blind trials suggest gabapentin might be useful in the treatment of pain. Doses ranging from 300-2400 mg/day were studied in treating diabetic neuropathy (Backonja, et al., 1998), postherpetic neuralgia (Rowbotham, et al., 1998), trigeminal neuralgia, migraine and pain associated with cancer and multiple sclerosis (Di Trapini, et al., 2000; Caraceni, et al., 1999; Houtchens, et al., 1997; see also Magnus, 1999; Laird & Gidal, 2000; Nicholson, 2000).

Ziconotide (Prialt®; SNX-111) is a synthetic analgesic derived from the cone snail peptide Conus magus MVIIA that has been shown to reversibly block N-type calcium channels. In a variety of animal models, the selective block of N-type channels via intrathecal administration of Ziconotide significantly depresses the formalin phase 2 response, thermal hyperalgesia, mechanical allodynia and post-surgical pain (Malmberg and Yaksh, 1994; Bowersox, et al., 1996; Sluka, 1998; Wang, et al., 1998).

Ziconotide has been evaluated in a number of clinical trials via intrathecal administration for the treatment of a variety of conditions including post-herpetic neuralgia, phantom limb syndrome, HIV-related neuropathic pain and intractable cancer pain (reviewed in Mathur, 2000). In phase II and III clinical trials with patients unresponsive to intrathecal opiates, Ziconotide has significantly reduced pain scores and in a number of specific instances resulted in relief after many years of continuous pain. Ziconotide is also being examined for the management of severe post-operative pain as well as for brain damage following stroke and severe head trauma (Heading, 1999). In two case studies Ziconotide has been further examined for usefulness in the management of intractable spasticity following spinal cord injury in patients unresponsive to baclofen and morphine (Ridgeway, et al., 2000). In one instance Ziconotide decreased the spasticity from the severe range to the mild to none range with few side effects. In another patient Ziconotide also reduced spasticity to the mild range although at the required dosage significant side effects including memory loss, confusion and sedation prevented continuation of the therapy.

T-type calcium channels are involved in various medical conditions. In mice lacking the gene expressing the α_{1G} subunit, resistance to absence seizures was observed (Kim, et al., 2001). Other studies have also implicated the α_{1H} subunit in the development of epilepsy (Su, et al., 2002). There is strong evidence that some existing anticonvulsant drugs, such as ethosuximide, function through the blockade of T-type channels (Gomora, et al., 2001).

Low voltage-activated calcium channels are highly expressed in tissues of the cardiovascular system. Mibefradil, a calcium channel blocker 10-30-fold selective for T-type over L-type channels, was approved for use in hypertension and angina. It was withdrawn from the market shortly after launch due to interactions with other drugs (Heady, et al., 2001).

Growing evidence suggests T-type calcium channels may also be involved in pain. Both mibefradil and ethosuximide have shown anti-hyperalgesic activity in the spinal nerve ligation model of neuropathic pain in rats (Dogrul, et al., 2003).

U.S. Pat. Nos. 6,011,035; 6,294,533; 6,310,059; and 6,492,375; PCT publications WO 01375 and WO 01/45709; PCT publications based on PCT CA 99/00612, PCT CA 00/01586; PCT CA 00/01558; PCT CA 00/01557; PCT CA 2004/000535; and PCT CA 2004/000539, and U.S. patent application Ser. Nos. 10/746,932 filed 23 Dec. 2003; Ser. No. 10/746,933 filed 23 Dec. 2003; Ser. No. 10/409,793 filed 8 Apr. 2003; Ser. No. 10/409,868 filed 8 Apr. 2003; Ser. No. 10/655,393 filed 3 Sep. 2003; Ser. No. 10/821,584 filed 9 Apr. 2004; and Ser. No. 10/821,389 filed 9 Apr. 2004 disclose calcium channel blockers where a piperidine or piperazine ring is substituted by various aromatic moieties. These applications and publications are incorporated herein by reference.

U.S. Pat. No. 5,646,149 describes calcium channel antagonists of the formula A-Y-B wherein B contains a piperazine or piperidine ring directly linked to Y. An essential component of these molecules is represented by A, which must be an antioxidant; the piperazine or piperidine itself is said to be important. The exemplified compounds contain a benzhydril substituent, based on known calcium channel blockers (see below). U.S. Pat. No. 5,703,071 discloses compounds said to be useful in treating ischemic diseases. A mandatory portion of the molecule is a tropolone residue, with substituents such as piperazine derivatives, including their benzhydril derivatives. U.S. Pat. No. 5,428,038 discloses compounds indicated to exhibit a neural protective and antiallergic effect. These compounds are coumarin derivatives which may include derivatives of piperazine and other six-membered heterocycles. A permitted substituent on the heterocycle is diphenylhydroxymethyl. Thus, approaches in the art for various indications which may involve calcium channel blocking activity have employed compounds which incidentally contain piperidine or piperazine moieties substituted with benzhydril but mandate additional substituents to maintain functionality.

Certain compounds containing both benzhydril moieties and piperidine or piperazine are known to be calcium channel antagonists and neuroleptic drugs. For example, Gould, R. J., et al., Proc Natl Acad Sci USA (1983) 80:5122-5125 describes antischizophrenic neuroleptic drugs such as lidoflazine, fluspirilene, pimozide, clopimozide, and penfluridol. It has also been shown that fluspirilene binds to sites on L-type calcium channels (King, V. K., et al., J Biol Chem (1989) 264:5633-5641) as well as blocking N-type calcium current (Grantham, C. J., et al., Brit J Pharmacol (1944) 111:483-488). In addition, Lomerizine, as developed by Kanebo KK, is a known calcium channel blocker. However, Lomerizine is not specific for N-type channels. A review of publications concerning Lomerizine is found in Dooley, D., Current Opinion in CPNS Investigational Drugs (1999) 1:116-125.

The foregoing publications are listed for convenience, and are not to be construed as prior art.

### DISCLOSURE OF THE INVENTION

The invention relates to compounds useful in treating conditions such as stroke, anxiety, overactive bladder, inflammatory bowel disease, irritable bowel syndrome, interstitial colitis, head trauma, migraine, chronic, neuropathic and acute pain, drug and alcohol addiction, neurodegenerative disorders, psychoses, sleep disorders, depression, epilepsy, diabetes, cancer, male contraception, hypertension, pulmonary hypertension, cardiac arrhythmias, congestive heart failure, angina pectoris and other indications associated with calcium metabolism, including synaptic calcium channel-mediated functions. The compounds of the invention are diarylamino derivatives of piperazine or amino piperidine with substituents that enhance the calcium channel blocking activity of the compounds. Thus, in one aspect, the invention is directed to compounds of the formula 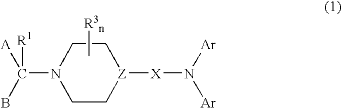
and salts or conjugates thereof,
wherein each of A and B is independently a 6-membered aromatic or nonaromatic, carbocyclic or heterocyclic moiety or is an aminoalkyl and wherein one and only one of A and B may be H or alkyl (1-8C);
R¹ is H or alkyl (1-8C);
Z is N or CHNR² wherein R² is H or alkyl (1-8C);
X is straight chain alkylene (1-4C) wherein a carbon adjacent to one nitrogen is in the form of C═O;
each R³ is independently a substituent selected from the group consisting of ═O, alkyl (1-8C), alkenyl (2-8C), alkynyl (2-8C), halo, CHF₂, CF₃, OCHF₂, OCF₃, CN, NO₂, NR₂, OR, SR, COR, COOR, CONR₂, NROCR, OOCR, SOR, SO₂R, SO₃R, SONR₂, SO₂NR₂, NRSOR, or NRSO₂R, wherein R is H or alkyl (1-8C), alkenyl (2-8C), alkynyl (2-8C), aryl and alkylaryl, and wherein two substituents on adjacent carbons may form an optionally substituted 5-7 membered ring;
n=0-2, and
Ar is a six-membered aromatic or heteroaromatic ring;
wherein each cyclic moiety included in A or B and each Ar moiety in formula (1) may be substituted by one or more substituents selected from the group consisting of ═O (in nonaromatic cyclic moieties), alkyl (1-6C), halo, CHF₂, CF₃, OCHF₂, OCF₃, NO₂, NR₂, OR, SR, COR, COOR, CONR₂, NROCR, OOCR, SOR, SO₂R, SO₃R, SONR₂, SO₂NR₂, NRSOR, and NRSO₂R, wherein R is H or alkyl (1-8C), alkenyl (2-8C), alkynyl (2-8C), aryl or alkylaryl, and
wherein two adjacent substituents may form a 5-7 membered ring, and wherein any alkyl, cyclic or aryl group recited above may itself be substituted by ═O, halo, CHF₂, CF₃, OCHF₂, OCF₃, NO₂, NR₂, OR, SR, COR, COOR, CONR₂, NROCR, OOCR, SOR, SO₂R, SO₃R, SONR₂, SO₂NR₂, NRSOR, or NRSO₂R, wherein R is H or alkyl (1-8C), alkenyl (2-8C), alkynyl (2-8C), aryl or alkylaryl.

The invention is also directed to methods to modulate calcium channel activity, preferably N-type and T-type channel activity, using the compounds of formula (1) and thus to treat certain undesirable physiological conditions; these conditions are associated with calcium channel activity. In another aspect, the invention is directed to pharmaceutical compositions containing these compounds, and to the use of these compounds for the preparation of medicaments for the treatment of conditions requiring modulation of calcium channel activity, including stroke, anxiety, overactive bladder, inflammatory bowel disease, irritable bowel syndrome, interstitial colitis, head trauma, migraine, chronic, neuropathic and acute pain, drug and alcohol addiction, neurodegenerative disorders, psychoses, sleep disorders, depression, epilepsy, diabetes, cancer, male contraception, hypertension, pulmonary hypertension, cardiac arrhythmias, congestive heart failure and angina pectoris.

### MODES OF CARRYING OUT THE INVENTION

The compounds of formula (1) useful in the methods of the invention exert their desirable effects through their ability to modulate the activity of N-type and/or T-type calcium channels. This makes them useful for treatment of certain conditions. Among such conditions where antagonist activity is desired are stroke, anxiety, epilepsy, head trauma, migraine, inflammatory bowel disease, overactive bladder irritable bowel syndrome, interstitial colitis and chronic, neuropathic and acute pain. Calcium flux is also implicated in other neurological disorders such as schizophrenia, anxiety, depression, other psychoses, neural degenerative disorders and drug and alcohol addiction and withdrawal. Other treatable conditions include cardiovascular conditions such as hypertension, pulmonary hypertension, congestive heart failure, angina pectoris and cardiac arrhythmias such as atrial fibrillation and ventricular fibrillation. In addition, T-type calcium channels have been implicated in certain types of cancer, diabetes, male contraception, sleep disorders and sexual dysfunction.

Chronic pain can include cancer pain, inflammatory pain conditions related to osteoarthritis, rheumatoid arthritis and fibromyalgia, and neuropathic pain. Neuropathic pain includes but is not limited to conditions such as diabetic peripheral neuropathy, post-herpetic neuralgia, trigeminal neuralgia, cancer pain and AIDS related neuropathy. Acute pain conditions can include nociceptive pain and post-operative pain.

Anxiety includes but is not limited to the following conditions: generalized anxiety disorder, social anxiety disorder, panic disorder, obsessive-compulsive disorder, and post-traumatic stress syndrome.

Neurodegenerative disorders include Parkinson's disease, Alzheimer's disease, multiple sclerosis, neuropathies, Huntington's disease and amyotrophic lateral sclerosis (ALS).

While the compounds of formula (1) generally have this activity, availability of this class of calcium channel modulators permits a nuanced selection of compounds for particular disorders. The availability of this class of compounds provides not only a genus of general utility in indications that are affected by calcium channel activity, but also provides a large number of compounds which can be mined and manipulated for specific interaction with particular forms of calcium channels. The availability of recombinantly produced calcium channels of the α_{1A}-α_{1I} and α_{1S} types set forth above, facilitates this selection process. Dubel, S. J., et al., Proc. Natl. Acad. Sci. USA (1992) 89:5058-5062; Fujita, Y., et al., Neuron (1993) 10:585-598; Mikami, A., et al., Nature (1989) 340:230-233; Mori, Y., et al., Nature (1991) 350:398-402; Snutch, T. P., et al., Neuron (1991) 7:45-57; Soong, T. W., et al., Science (1993) 260:1133-1136; Tomlinson, W. J., et al., Neuropharmacology (1993) 32:1117-1126; Williams, M. E., et al., Neuron (1992) 8:71-84; Williams, M. E., et al., Science (1992) 257:389-395; Perez-Reyes, et al., Nature (1998) 391:896-900; Cribbs, L. L., et al., Circulation Research (1998) 83:103-109; Lee, J. H., et al., Journal of Neuroscience (1999) 19:1912-1921; McRory, J. E., et al., Journal of Biological Chemistry (2001) 276:3999-4011.

It is known that calcium channel activity is involved in a multiplicity of disorders, and particular types of channels are associated with particular conditions. The association of N-type and T-type channels in conditions associated with neural transmission would indicate that compounds of the invention which target N-type channels are most useful in these conditions. Many of the members of the genus of compounds of formula (1) exhibit high affinity for N-type channels and/or T-type channels. Thus, as described below, they are screened for their ability to interact with N-type and/or T-type channels as an initial indication of desirable function. It is desirable that the compounds exhibit IC₅₀ values of <1 μM. The IC₅₀ is the concentration which inhibits 50% of the calcium, barium or other permeant divalent cation flux at a particular applied potential.

There are three distinguishable types of calcium channel inhibition. The first, designated “open channel blockage,” is conveniently demonstrated when displayed calcium channels are maintained at an artificially negative resting potential of about −100 mV (as distinguished from the typical endogenous resting maintained potential of about −70 mV). When the displayed channels are abruptly depolarized under these conditions, calcium ions are caused to flow through the channel and exhibit a peak current flow which then decays. Open channel blocking inhibitors diminish the current exhibited at the peak flow and can also accelerate the rate of current decay.

This type of inhibition is distinguished from a second type of block, referred to herein as “inactivation inhibition.” When maintained at less negative resting potentials, such as the physiologically important potential of −70 mV, a certain percentage of the channels may undergo conformational change, rendering them incapable of being activated—i.e., opened—by the abrupt depolarization. Thus, the peak current due to calcium ion flow will be diminished not because the open channel is blocked, but because some of the channels are unavailable for opening (inactivated). “Inactivation” type inhibitors increase the percentage of receptors that are in an inactivated state.

A third type of inhibition is designated “resting channel block”. Resting channel block is the inhibition of the channel that occurs in the absence of membrane depolarization, that would normally lead to opening or inactivation. For example, resting channel blockers would diminish the peak current amplitude during the very first depolarization after drug application without additional inhibition during the depolarization.

In order to be maximally useful in treatment, it is also helpful to assess the side reactions which might occur. Thus, in addition to being able to modulate a particular calcium channel, it is desirable that the compound has very low activity with respect to the HERG K⁺ channel which is expressed in the heart. Compounds that block this channel with high potency may cause reactions which are fatal. Thus, for a compound that modulates the calcium channel, it should also be shown that the HERG K⁺ channel is not inhibited. Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow.

The Invention Compounds

The substituents on the basic structures of formula (1) are described above. These include alkyl, alkenyl, alkynyl, etc., substituents.

As used herein, the term “alkyl,” “alkenyl” and “alkynyl” include straight-chain, branched-chain and cyclic monovalent substituents, containing only C and H when they are unsubstituted or unless otherwise noted. Examples include methyl, ethyl, isobutyl, cyclohexyl, cyclopentylethyl, 2-propenyl, 3-butynyl, and the like. Typically, the alkyl, alkenyl and alkynyl substituents contain 1-10C or 1-8C (alkyl) or 2-1° C. or 2-8C (alkenyl or alkynyl). Preferably they contain 1-6C or 1-4C (lower alkyl) or 2-6C or 2-4C (lower alkenyl or lower alkynyl).

Heteroalkyl, heteroalkenyl and heteroalkynyl are similarly defined but may contain one or more O, S or N heteroatoms or combinations thereof within the backbone residue.

As used herein, “acyl” encompasses the definitions of alkyl, alkenyl, alkynyl, each of which is coupled to an additional residue through a carbonyl group. Heteroacyl includes the related heteroforms.

“Aromatic” moiety or “aryl” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroarylalkyl” refer to aromatic and heteroaromatic systems which are coupled to another residue through a carbon chain, including substituted or unsubstituted, saturated or unsaturated, carbon chains, typically of 1-8C, or the hetero forms thereof. These carbon chains may also include a carbonyl group, thus making them able to provide substituents as an acyl or heteroacyl moiety.

In general, any alkyl, alkenyl, alkynyl, acyl, or aryl (including the heteroforms) group contained in a substituent may itself optionally be substituted by additional substituents. The nature of these substituents is similar to those recited with regard to the primary substituents themselves. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments. However, alkyl substituted by aryl, amino, alkoxy, and the like would be included.

Non-interfering substituents in general include, but are not limited to, alkyl, alkenyl, alkynyl, aryl, arylalkyl, acyl, ═O, halo, OR, NR₂, SR, —SOR, —SO₂R, SO₂R, —OCOR, —NRCOR, —NRCONR₂, —NRCOOR, —OCONR₂, —RCO, —COOR, NRSOR, NRSO₂R, —CONR₂, SONR₂, and/or SO₂NR₂ (wherein each R is independently H or alkyl (1-8C), alkenyl(2-8C), alkynyl(2-8C), aryl or arylalkyl), —CN, —CF₃, and NO₂, and like substituents.

In the compounds of the invention, Ar is preferably optionally substituted phenyl, 2-, 3- or 4-pyridyl, indolyl, 2- or 4-pyrimidyl, pyridazinyl, benzotriazolyl or benzimidazolyl. More preferably Ar is phenyl, pyridyl, or pyrimidyl. Most preferably Ar is phenyl. Each of these embodiments may optionally be substituted with one or more groups defined above, such as alkyl, alkenyl, alkynyl, aryl, O-aryl, O-alkylaryl, O-aroyl, NR-aryl, N-alkylaryl, NR-aroyl, halo, OR, NR₂, SR, —OOCR, —NROCR, RCO, —COOR, —CONR₂, and/or SO₂NR₂, wherein each R is independently H or alkyl (1-8C), alkenyl (2-8C), alkynyl (2-8C), aryl or alkylaryl, and/or by —CN, —CF₃, and/or NO₂. Alkyl, alkenyl, alkynyl, cyclic and aryl portions of these may be further substituted by similar substituents.

Among preferred substituents on Ar are alkyl, CF₃, CHF₂, OR, SR, NR₂, where R is as above-defined, and halo. Preferred embodiments of R¹ are methyl and H. Preferred embodiments of R³ include=0 and carboxy.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Suitable pharmaceutically acceptable acids and bases are well-known in the art, such as hydrochloric, sulphuric, citric, acidic, or tartaric acids and potassium hydroxide, sodium hydroxide, ammonium hydroxide, caffeine, various amines, and the like. Methods for preparation of the appropriate salts are well-established in the art.

In some cases, the compounds of the invention contain one or more chiral centers. The invention includes the isolated stereoisomeric forms as well as mixtures of stereoisomers in varying degrees of chiral purity.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

Synthesis of the Invention Compounds

The compounds of the invention may be synthesized using conventional methods.

Reaction Scheme 1 is illustrative and may be used to prepare compounds with piperazine rings adjacent C═O. The piperidine analog can be substituted and reaction of the nitrogen of CHNH₂ substitutes for the nitrogen of piperazine. Also shown is the pathway where an alkylene component (e.g., CH₂>is adjacent the piperazine or the N of CHNH of piperidine.

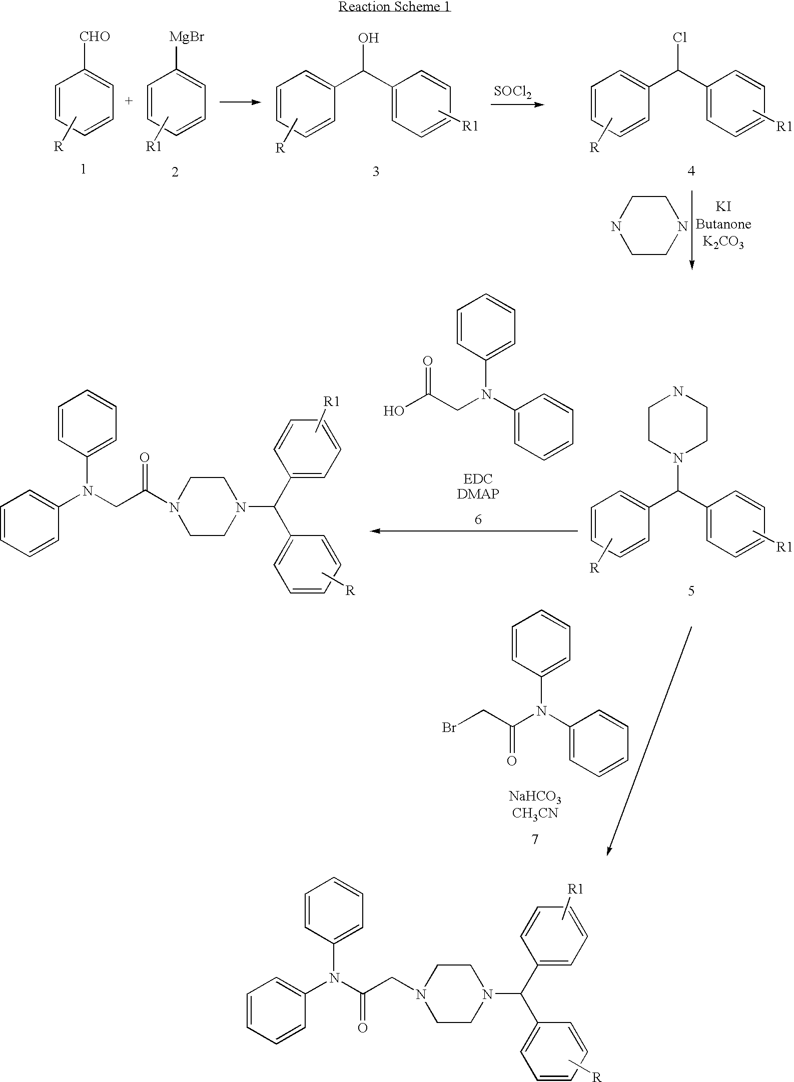

The synthesis is illustrated for the case wherein, in formula (1), R¹ is H, and a and b are both optionally substituted phenyl, Z is N, X is CH₂CO, and both Ar are unsubstituted phenyl. It will be clear that the same scheme will apply for the remaining embodiments permitted for these named substituents. In the first step, compounds 1 and 2 are coupled through a Grignard reaction to form the diphenyl methanol which is then converted to the diphenyl methyl chloride. The resulting compound 4 is then treated with piperazine (or, in the alternative, 4-amino piperidine to obtain compound 5. Compound 5 is then reacted with diphenylamino acetic acid under conditions whereby an amide is formed with the unsubstituted piperazine ring nitrogen or the 4-amino group of piperidine. Alternatively, the compound of formula (5) is reacted with an α-brominated form of the amide 7 to obtain the desired compound as shown. If Z is CHNR², the 4-amino group substitutes for the nitrogen of the piperazine in this reaction.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, i.e., the N-type channel. In addition, using standard screening protocols, the libraries may be screened for compounds which block additional channels or receptors such as sodium channels, potassium channels and the like.

Methods of performing these screening functions are well known in the art. These methods can also be used for individually ascertaining the ability of a compound to agonize or antagonize the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) may be used alone, as mixtures of two or more compounds of formula (1) or in combination with other pharmaceuticals. Depending on the mode of administration, the compounds will be formulated into suitable compositions to permit facile delivery.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.1-15 mg/kg, preferably 0.1-1 mg/kg. However, dosage levels are highly dependent on the nature of the condition, drug efficacy, the condition of the patient, the judgment of the practitioner, and the frequency and mode of administration.

The following examples are intended to illustrate but not to limit the invention.

### EXAMPLE 1

### Synthesis of 1-{4-[(4-Chloro-phenyl)-phenyl-methyl]-piperazin-1-yl}-2-diphenylaminoethanone

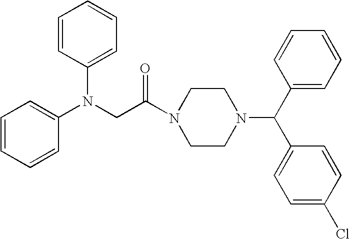

### A. Synthesis of (4-Chloro-phenyl)-phenyl-methanol

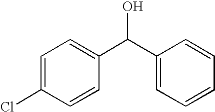

A solution of 4-chlorobenzaldehyde (1.03 g, 7.34 mmol) in dry ether (10 ml) was added slowly to a solution of phenylmagnesium bromide (2.3 ml, 6.98 mmol, 3.0 M in ether) under nitrogen. The mixture was heated to reflux for 1 hour then cooled to 0° C. and hydrolyzed with 1 N HCl (40 ml). The aqueous phase was extracted with ether (3×) and combined organic layer dried over MgSO₄. The crude product was purified using hexane:ethyl acetate (5:1) as eluant to give 1.5 g of pure product.

### B. Synthesis of 1-Chloro-4-(chloro-phenyl-methyl)-benzene

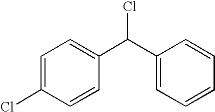

To a solution of (4-chloro-phenyl)-phenyl-methanol (2.41 g, 11.06 mmol) in dry benzene (20 ml) was added SOCl₂ (8.25 ml, 10 mmol) and anhydrous CaCl₂ (2 g). The mixture was heated under reflux for 2 hours and then cooled and stirred at r.t. overnight. It was then filtered and solvent removed in vacuo to give a pale yellow oil and was used in the next step without further purification.

### C. Synthesis of 1-[(4-Chloro-phenyl)-phenyl-methyl]-piperazine

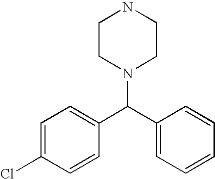

A mixture of 1-chloro-4-(chloro-phenyl-methyl)-benzene (4.12 g, 17.4 mmol) in butanone (20 ml), anhydrous piperazine (5.98 g, 69.6 mmol), anhydrous K₂CO₃ (2.40 g, 17.4 mmol) and KI (2.88 g, 17.4 mmol) was refluxed under nitrogen for 18 hours. The mixture was then cooled and filtered and the solvent removed in vacuo. The residue was dissolved in CH₂Cl₂ (100 ml) and washed with water (30 ml). Drying and removal of the solvent followed by chromatography (CH₂Cl₂: CH₃OH: NH₄OH 90:10:0.5>afforded desired product in 57% yield.

### D. Synthesis of Final Product

To a solution of 1-[(4-chloro-phenyl)-phenyl-methyl]-piperazine (0.59 g, 2.08 mmol) in dry CH₂Cl₂ (40 ml) was added diphenylaminoacetic acid (0.472 g, 2.08 mmol) under nitrogen. To the reaction was added EDC (0.797 g, 4.16 mmol) and DMAP (cat) and the reaction mixture stirred under nitrogen at room temperature overnight. The reaction was then concentrated under reduced pressure. The residue dissolved in ethyl acetate: water (10:1) (150 ml). The organic was washed with water (30 ml, 2×) and 10% NaOH (30 ml) and dried over MgSO₄ and evaporated to dryness. The resulting residue was purified by column chromatography using hexane:ethyl acetate (3:1) to give desired product in 76% yield.

### EXAMPLE 2

### Synthesis of 2-Diphenylamino-1-[4-(phenyl-pyridin-4-yl-methyl)-piperazin-1-yl]-ethanone

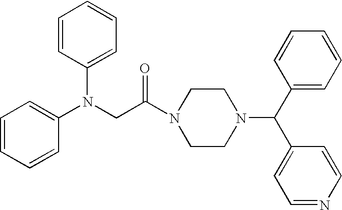

To a solution of 1-(phenyl-pyridin-4-yl-methyl]-piperazine (0.58 g, 2.29 mmol) in dry CH₂Cl₂ (40 ml) was added diphenylaminoacetic acid (0.51 g, 2.29 mmol) under nitrogen. To the reaction was added EDC (0.878 g, 4.58 mmol) and DMAP (cat) and the reaction mixture stirred under nitrogen at room temperature overnight. The reaction was then concentrated under reduced pressure. The residue dissolved in ethyl acetate: water (10:1) (150 ml). The organic was washed with water (30 ml, 2×) and 10% NaOH (30 ml) and dried over MgSO₄ and evaporated to dryness. The resulting residue was purified by column chromatography using hexane:ethyl acetate (1:1) to give desired product in 79% yield.

### EXAMPLE 3

### Synthesis of 2-(4-Benzhydryl-piperazin-1-yl)-N,N-diphenyl-acetamide

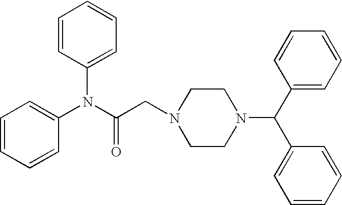

To a solution of diphenylmethyl piperazine (0.6 g, 2.37 mmol) in dry CH₃CN (20 ml) was added 2-bromo-N,N-diphenyl acetamide (0.68 g, 2.37 mmol) and NaHCO₃ (0.4 g, 4.74 mmol) under nitrogen. The reaction mixture was refluxed over night. After cooling, the solvent was evaporated and residue was taken up with water (15 ml) and extracted with CHCl₃ (3×50 ml). The organic was dried over MgSO₄ and evaporated to dryness. The resulting residue was purified by column chromatography using hexane:ethyl acetate (2:1) to give the desired product in 84% yield.

### EXAMPLE 4

### Synthesis of 2-[4-(1-Methyl-piperidin-4-ylmethyl)-piperazin-1-yl]-N,N-diphenyl-acetamide

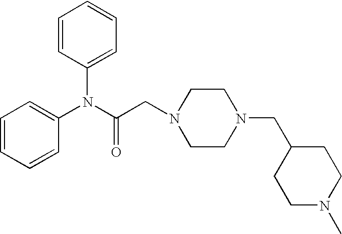

To a solution of 1-(1-methyl-piperidin-4ylmethyl)-piperazine (0.5 g, 2.7 mmol) in dry CH₃CN (20 ml) was added 2-bromo-N,N-diphenyl acetamide (0.61 g, 2.7 mmol) and NaHCO₃ (0.45 g, 5.4 mmol) under nitrogen. The reaction mixture was refluxed over night. After cooling, the solvent was evaporated and residue was taken up with water (15 ml) and extracted with CHCl₃ (3×50 ml); The organic was dried over MgSO₄ and evaporated to dryness. The resulting residue was purified by column chromatography using CH₂Cl₂:CH₃OH (10:1) to give the desired product in 80% yield.

### EXAMPLE 5

### Summary of Compounds Prepared

Following the general procedures set forth above, the following compounds were prepared:

| Compound | | |
|---|---|---|
| No. | Name | Structure |
| 1 | 1-(4-Benzhydryl-piperazin-1-yl)- 2-diphenylamino-ethanone | 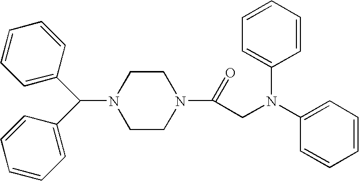 |
| 2 | 1-{4-[(2,4-Dimethyl-phenyl)- phenyl-methyl]-piperazin-1-yl}- 2-diphenylamino-ethanone | 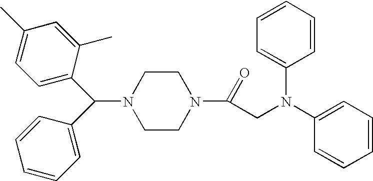 |
| 3 | 1-{4-[(2,4-Dichloro-phenyl)- phenyl-methyl]-piperazin-1-yl}- 2-diphenylamino-ethanone | 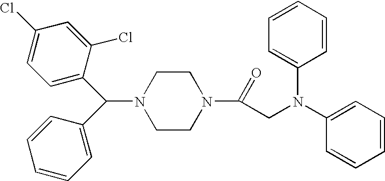 |
| 4 | 1-{4-[(4-Chloro-phenyl)-phenyl- methyl]-piperazin-1-yl}-2- diphenylamino-ethanone | 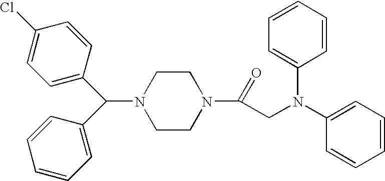 |
| 5 | 1-{4-[(3-Chloro-phenyl)-phenyl- methyl]-piperazin-1-yl}-2- diphenylamino-ethanone | 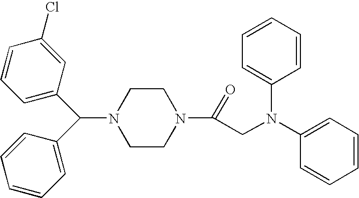 |
| 6 | 1-{4-[(2-Chloro-phenyl)-phenyl- methyl]-piperazin-1-yl}-2- diphenylamino-ethanone | 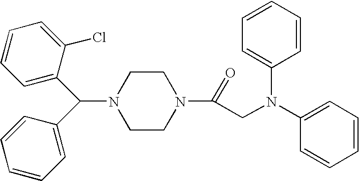 |
| 7 | 1-{4-[(2,3-Dichloro-phenyl)- phenyl-methyl]-piperazin-1-yl}- 2-diphenylamino-ethanone | 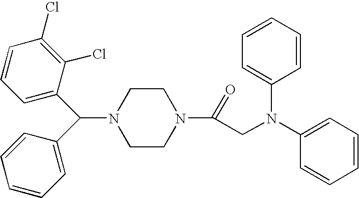 |
| 8 | 1-[4-(Benzo[1,3]dioxol-5-yl- phenyl-methyl)-piperazin-1-yl]-2- diphenylamino-ethanone | 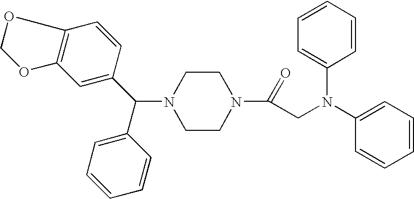 |
| 9 | 2-Diphenylamino-1-{4-[(4- methoxy-phenyl)-(4- trifluoromethyl-phenyl)-methyl]- piperazin-1-yl}-ethanone | 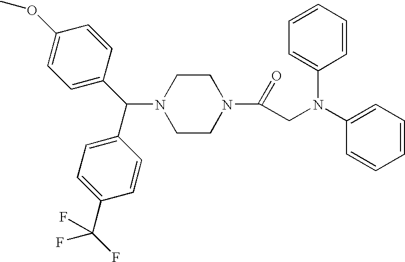 |
| 10 | 2-Diphenylamino-1-[4-(1-methyl- piperidin-3-ylmethyl)-piperazin- 1-yl]-ethanone | 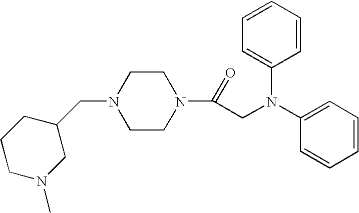 |
| 11 | 2-Diphenylamino-1-(4-pyridin-3- ylmethyl-piperazin-1-yl)- ethanone | 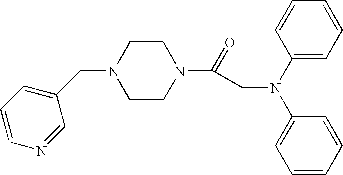 |
| 12 | 2-Diphenylamino-1-(4-pyridin-2- ylmethyl-piperazin-1-yl)- ethanone | 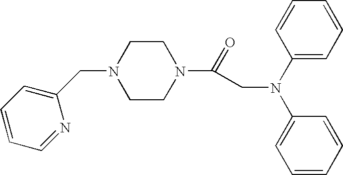 |
| 13 | 2-Diphenylamino-1-[4-(phenyl- pyridin-3-yl-methyl)-piperazin-1- yl]-ethanone | 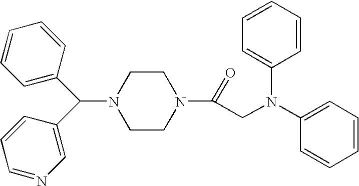 |
| 14 | 2-Diphenylamino-1-[4-(phenyl- pyridin-2-yl-methyl)-piperazin-1- yl]-ethanone | 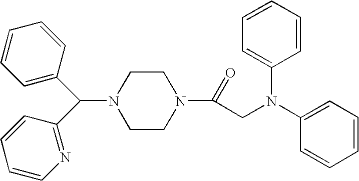 |
| 15 | 1-{4-[(4-tert-Butyl-phenyl)- phenyl-methyl]-piperazin-1-yl}- 2-diphenylamino-ethanone | 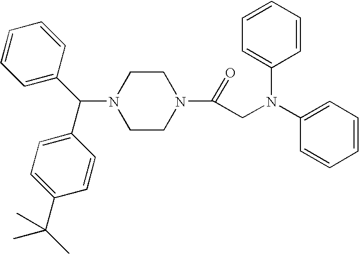 |
| 16 | 2-(4-Benzhydryl-piperazin-1-yl)- N,N-diphenyl-acetamide | 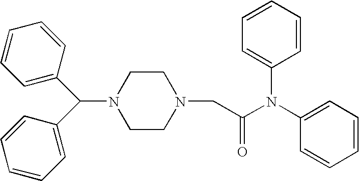 |
| 17 | 2-{4-[(2,4-Dichloro-phenyl)- phenyl-methyl]-piperazin-1-yl}- N,N-diphenyl-acetamide | 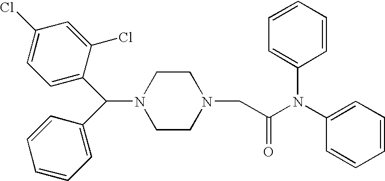 |
| 18 | 2-{4-[(2,4-Dimethyl-phenyl)- phenyl-methyl]-piperazin-1-yl}- N,N-diphenyl-acetamide | 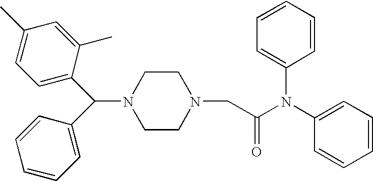 |
| 19 | 2-{4-[(4-Chloro-phenyl)-phenyl- methyl]-piperazin-1-yl}-N,N- diphenyl-acetamide | 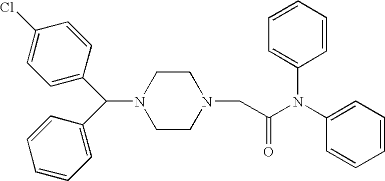 |
| 20 | 2-{4-[(3-Chloro-phenyl)-phenyl- methyl]-piperazin-1-yl}-N,N- diphenyl-acetamide | 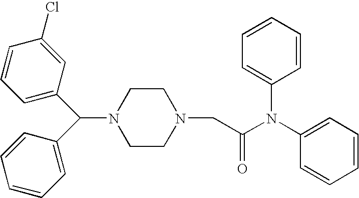 |
| 21 | 2-{4-[(2-Chloro-phenyl)-phenyl- methyl]-piperazin-1-yl}-N,N- diphenyl-acetamide | 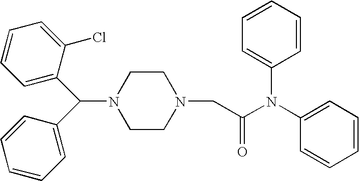 |
| 22 | 2-{4-[(2,3-Dichloro-phenyl)- phenyl-methyl]-piperazin-1-yl}- N,N-diphenyl-acetamide | 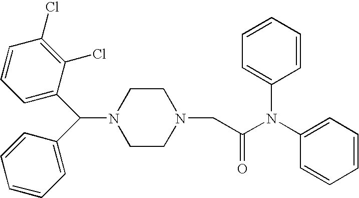 |
| 23 | 2-[4-(Benzo[1,3]dioxol-5-yl- phenyl-methyl)-piperazin-1-yl]- N,N-diphenyl-acetamide | 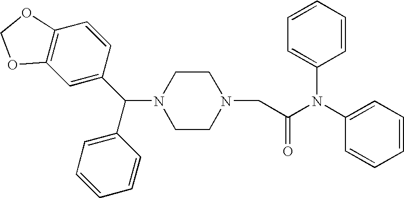 |
| 24 | 2-{4-[(4-Methoxy-phenyl)-(4- trifluoromethyl-phenyl)-methyl]- piperazin-1-yl}-N,N-diphenyl- acetamide | 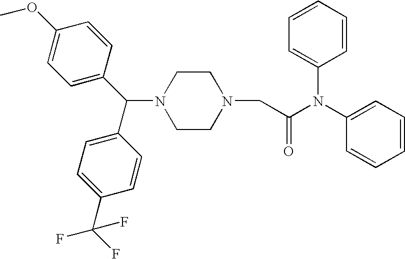 |
| 25 | 2-[4-(1-Methyl-piperidin-4- ylmethyl)-piperazin-1-yl]-N,N- diphenyl-acetamide | 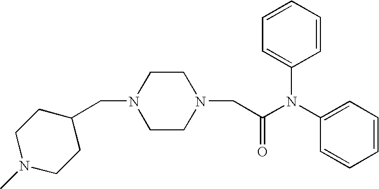 |
| 26 | 2-[4-(3-Dimethylamino-propyl)- piperazin-1-yl]-N,N-diphenyl- acetamide | 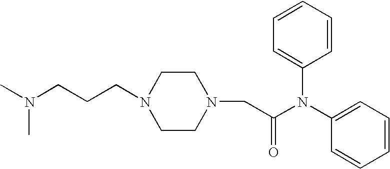 |
| 27 | 2-[4-(1-Methyl-piperidin-3- ylmethyl)-piperazin-1-yl]-N,N- diphenyl-acetamide | 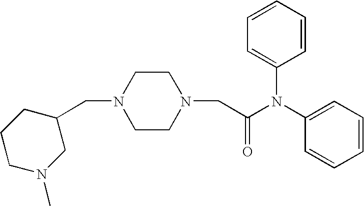 |
| 28 | N,N-Diphenyl-2-[4-(phenyl- pyridin-3-yl-methyl)-piperazin-1- yl]-acetamide | 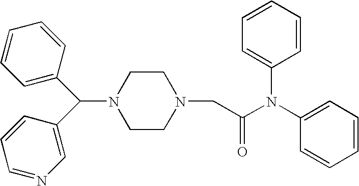 |
| 29 | N,N-Diphenyl-2-[4-(phenyl- pyridin-2-yl-methyl)-piperazin-1- yl]-acetamide | 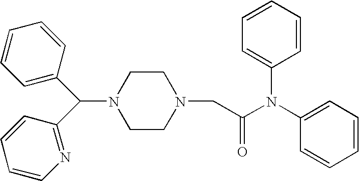 |
| 30 | 2-{4-[(4-tert-Butyl-phenyl)- phenyl-methyl]-piperazin-1-yl}- N,N-diphenyl-acetamide | 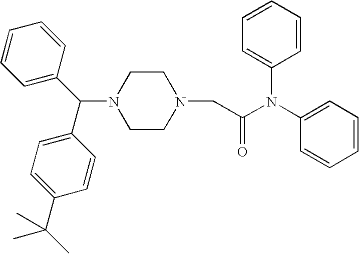 |
| 31 | 2-{4-[(4-Methoxy-phenyl)- phenyl-methyl]-piperazin-1-yl}- N,N-diphenyl-acetamide | 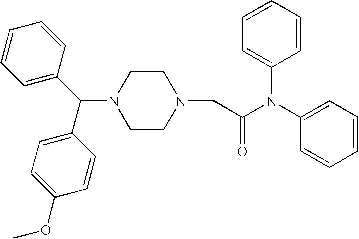 |
| 32 | 2-(4-Benzhydryl-2,3-dioxo- piperazin-1-yl)-N,N-diphenyl- acetamide | 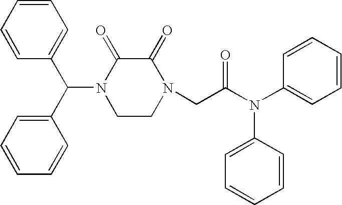 |
| 33 | 2-(4-Benzhydryl-2,5-dioxo- piperazin-1-yl)-N,N-diphenyl- acetamide | 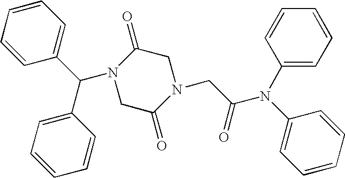 |
| 34 | 1-Benzhydryl-4-(2- diphenylamino-acetyl)- piperazine-2,5-dione | 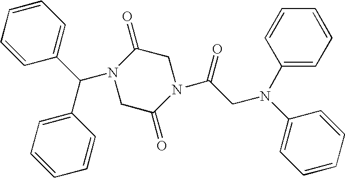 |

Also prepared are the foregoing compounds wherein the nitrogen of piperazine coupled through Z to diphenyl amine is substituted by CHNH.

### EXAMPLE 6

N-type Channel Blocking Activities of Various Invention Compounds

A. Transformation of HEK cells:

N-type calcium channel blocking activity was assayed in human embryonic kidney cells, HEK 293, stably transfected with the rat brain N-type calcium channel subunits (α_{1B}+α₂δ+β_{1b} cDNA subunits). Alternatively, N-type calcium channels (α_{1B}+α₂δ+β_{1b} cDNA subunits), L-type channels (α_{1C}+δ+β_{1b} cDNA subunits) and P/Q-type channels (α_{1A}+α₂δ+β_{1b} cDNA subunits) were transiently expressed in HEK 293 cells. Briefly, cells were cultured in Dulbecco's modified eagle medium (DMEM) supplemented with 10% fetal bovine serum, 200 U/ml penicillin and 0.2 mg/ml streptomycin at 37° C. with 5% CO₂. At 85% confluency cells were split with 0.25% trypsin/l mM EDTA and plated at 10% confluency on glass coverslips. At 12 hours the medium was replaced and the cells transiently transfected using a standard calcium phosphate protocol and the appropriate calcium channel cDNA's. Fresh DMEM was supplied and the cells transferred to 28° C./5% CO₂. Cells were incubated for 1 to 2 days before cell recording.

B. Measurement of Inhibition

Whole cell patch clamp experiments were performed using an Axopatch 200B amplifier (Axon Instruments, Burlingame, Calif.) linked to a personal computer equipped with PCLAMP software. The external and internal recording solutions contained, respectively, 5 mM BaCl₂, 10 mM MgCl₂, 10 mM HEPES, 40 mM TEACl, 10 mM glucose, 87.5 mM CsCl (pH 7.2) and 108 mM CsMS, 4 mM MgCl₂, 9 mM EGTA, 9 mM HEPES (pH 7.2). Currents were typically elicited from a holding potential of −80 mV to +10 mV using Clampex software (Axon Instruments). Typically, currents were first elicited with low frequency stimulation (0.067 Hz) and allowed to stabilize prior to application of the compounds. The compounds were then applied during the low frequency pulse trains for two to three minutes to assess tonic block, and subsequently the pulse frequency was increased to 0.2 Hz to assess frequency dependent block. Data were analyzed using Clampfit (Axon Instruments) and SigmaPlot 4.0 (Jandel Scientific).

Specific data obtained for N-type channels are shown in Table 1 below. See Example 5 for structures.

TABLE 1
N-type Calcium Channel Block

| Compound | IC₅₀ @ 0.067 Hz (μM) | IC₅₀ @ 0.2 Hz (μM) |
|---|---|---|
| 1 | 6.63 | 6.61 |
| 2 | 0.29 | 0.26 |
| 3 | 0.55 | 0.46 |
| 4 | 0.55 | 0.50 |
| 5 | 0.31 | 0.28 |
| 6 | 0.33 | 0.32 |
| 7 | 0.18 | 0.14 |
| 10 | 1.10 | 0.98 |
| 16 | 0.29 | 0.26 |
| 17 | 0.83 | 0.51 |
| 18 | 1.76 | 1.25 |
| 19 | 0.19 | 0.11 |
| 20 | 0.34 | 0.32 |
| 21 | 0.124 | 0.052 |
| 22 | 0.30 | 0.24 |
| 25 | 0.46 | 0.46 |
| 26 | 0.35 | 0.34 |

### EXAMPLE 7

### T-Type Channel Blocking Activities of Various Invention Compounds

Standard patch-clamp techniques were employed to identify blockers of T-type currents. Briefly, previously described HEK cell lines stably expressing human α_{1G} T-type channels were used for all the recordings (passage #: 4-20, 37° C., 5% CO₂). To obtain T-type currents, plastic dishes containing semi-confluent cells were positioned on the stage of a ZEISS AXIOVERT S100 microscope after replacing the culture medium with external solution (see below). Whole-cell patches were obtained using pipettes (borosilicate glass with filament, O.D.: 1.5 mm, I.D.: 0.86 mm, 10 cm length), fabricated on a SUTTER P-97 puller with resistance values of ˜5 MΩ (see below for internal solution).

TABLE 2
External Solution 500 ml - pH 7.4, 265.5 mOsm

| Salt | Final mM | Stock M | Final ml | |
|---|---|---|---|---|
| CsCl | 132 | 1 | 66 | |
| CaCl₂ | 2 | 1 | 1 | |
| MgCl₂ | 1 | 1 | 0.5 | |
| HEPES | 10 | 0.5 | 10 | |
| glucose | 10 | — | 0.9 | grams |

TABLE 3
Internal Solution 50 ml - pH 7.3 with CsOH, 270 mOsm

| Salt | Final mM | Stock M | Final ml | |
|---|---|---|---|---|
| Cs-Methanesulfonate | 108 | — | 1.231 | gr/50 ml |
| MgCl2 | 2 | 1 | 0.1 | |
| HEPES | 10 | 0.5 | 1 | |
| EGTA-Cs | 11 | 0.25 | 2.2 | |
| ATP | 2 | 0.2 | 0.025 | |

(1 aliquot/2.5 ml)
T-type currents were reliably obtained by using two voltage protocols:
(1) “non-inactivating”, and
(2) “inactivation”

In the non-inactivating protocol, the holding potential is set at −110 mV and with a pre-pulse at −100 mV for 1 second prior to the test pulse at 40 mV for 50 ms. In the inactivation protocol, the pre-pulse is at approximately −85 mV for 1 second, which inactivates about 15% of the T-type channels.

[image]

Test compounds were dissolved in external solution, 0.1-0.01% DMSO. After ˜10 min rest, they were applied by gravity close to the cell using a WPI microfil tubing. The “non-inactivated” pre-pulse was used to examine the resting block of a compound. The “inactivated” protocol was employed to study voltage-dependent block. However, the initial data shown below were mainly obtained using the non-inactivated protocol only. IC₅₀ values are shown for various compounds of the invention in Table 4.

TABLE 4
T-type Calcium Channel Block

| Compound | IC₅₀ - 100 mV (μM) | IC₅₀ - 80 mV (μM) |
|---|---|---|
| 1 | 1.35 | 0.51 |
| 2 | 2.00 | 0.444 |
| 3 | no effect | 6.00 |
| 4 | 3.80 | 0.748 |
| 5 | no effect | 0.80 |
| 6 | no effect | 1.20 |
| 7 | 1.00 | 1.00 |
| 10 | 10.00 | 2.00 |
| 16 | no effect | 1.30 |
| 17 | 0.118 | 0.029 |
| 18 | 0.229 | 0.084 |
| 19 | 0.072 | 0.022 |
| 20 | 0.114 | 0.033 |
| 21 | 0.134 | 0.034 |
| 22 | 0.081 | 0.016 |
| 25 | 5.70 | 3.70 |
| 26 | no effect | 1.00 |

### EXAMPLE 8

### Activity of Invention Compounds in Formalin-Induced Pain Model

The effects of intrathecally delivered compounds of the invention on the rat formalin model were measured. The compounds were reconstituted to stock solutions of approximately 10 mg/ml in propylene glycol. Eight Holtzman male rats of 275-375 g size were randomly selected per test article.

The following study groups were used, with test article, vehicle control (propylene glycol) and saline delivered intraperitoneally (IP):

TABLE 5
Formalin Model Dose Groups

| Test/Control Article | Dose | Route | Rats per group |
|---|---|---|---|
| Compound | 30 mg/kg | IP | 6 |
| Propylene glycol | N/A | IP | 4 |
| Saline | N/A | IP | 7 |

N/A = Not Applicable

Prior to initiation of drug delivery baseline behavioral and testing data were taken. At selected times after infusion of the Test or Control Article these data were again collected.

On the morning of testing, a small metal band (0.5 g) was loosely placed around the right hind paw. The rat was placed in a cylindrical Plexiglas chamber for adaptation a minimum of 30 minutes. Test Article or Vehicle Control Article was administered 10 minutes prior to formalin injection (50 μl of 5% formalin) into the dorsal surface of the right hindpaw of the rat. The animal was then placed into the chamber of the automated formalin apparatus where movement of the formalin injected paw was monitored and the number of paw flinches tallied by minute over the next 60 minutes (Malmberg, A. B., et al., Anesthesiology (1993) 79:270-281).

Results are presented as Maximum Possible Effect±SEM, where saline control=100%.

| Compound | Phase I | Phase II Total | Phase IIA | Phase IIB |
|---|---|---|---|---|
| 7 | 60 ± 7 | 63 ± 10 | 63 ± 13 | 64 ± 11 |

### EXAMPLE 9

### Spinal Nerve Ligation Model of Neuropathic Pain

Spinal nerve ligation (SNL) injury was induced using the procedure of Kim and Chung, (Kim and Chung 1992) in male Sprague-Dawley rats (Harlan; Indianapolis, Ind.) weighing 200 to 300 grams. Anesthesia was induced with 2% halothane in O₂ at 2 L/min and maintained with 0.5% halothane in O₂. After surgical preparation of the rats and exposure of the dorsal vertebral column from L₄ to S₂, the L₅ and L₆ spinal nerves were tightly ligated distal to the dorsal root ganglion using 4-0 silk suture. The incision was closed, and the animals were allowed to recover for 5 days. Rats that exhibited motor deficiency (such as paw-dragging) or failure to exhibit subsequent tactile allodynia were excluded from further testing. Sham control rats underwent the same operation and handling as the experimental animals, but without SNL.

The method of Hargreaves and colleagues (Hargreaves, et al., 1988) was employed to access paw-withdrawal latency to a thermal nociceptive stimulus. Rats were allowed to acclimate within a plexiglas enclosure on a clear glass plate maintained at 30° C. A radiant heat source (i.e., high intensity projector lamp) was activated with a timer and focused onto the plantar surface of the affected paw of nerve-injured or carrageenan-injected rats. Paw-withdrawal latency was determined by a photocell that halted both lamp and timer when the paw was withdrawn. The latency to withdrawal of the paw from the radiant heat source was determined prior to carrageenan or L5/L5 SNL, 3 hours after carrageenan or 7 days after L5/L6 SNL but before drug and after drug ministration. A maximal cut-off of 40 seconds was Employed to prevent tissue damage. Paw withdrawal latencies were thus determined to the Nearest 0.1 second. Reversal of thermal hyperalgesia was indicated by a return of the paw Withdrawal latencies to the pre-treatment baseline latencies (i.e., 21 seconds). Anti nociception Was indicated by a significant (p <0.05) increase in paw withdrawal latency above this baseline. Data were converted to % anti hyperalgesia by the formula: (100qx (test latency−baseline latency)/(cut-off−baseline latency) where cut-off was 21 seconds for determining anti hyperalgesia.

Compound 7 was administered orally in propylene glycol solution at a dose of 30 mg/kg. The percent activity was calculated for thermal hyperalgesia.

TABLE 6
% Activity in SNL Model of Neuropathic Pain

| | Thermal Hyperalgesia |
|---|---|
| Time (min) | (% Activity) |
| 30 | 38.45 ± 18.52 |
| 60 | 78.54 ± 10.74 |
| 90 | 40.21 ± 17.08 |
| 120 | 20.98 ± 10.36 |

### REFERENCES

Augustine, G. J., Chariton, M. P. & Smith, S. J. (1987) Calcium action in synaptic transmitter release. Annu Rev Neurosci 10: 633-693.
Backonja, M., Beydoun, A, Edwards, K. R., Schwartz, S. L. Fonesca, V., Hes, M., LaMoreaux, L. & Garofalo, E. (1998) Gabapentin for the symptomatic treatment of painful neuropathy in patients with diabetes mellitus. JAMA 280: 1831-1836.
Bourinet E., Soong T. W., Sutton K., Slaymaker S., Mathews E., Monteil A., Zamponi G. W., Nargeot J. & Snutch T. P. (1999) Splicing of alpha 1A subunit gene generates phenotypic variants of P- and Q-type calcium channels. Nature Neuroscience 2: 407-415.
Bowersox, S. S., Gadbois, T., Singh, T., Pettus, M., Wang, Y-X & Luther, R. R. (1996) Selective N-type neuronal voltage-sensitive calcium channel blocker. SNX-111, produces spinal anticiception in rat models of acute, persistent and neuropathic pain. J Pharmacol Exp Ther 279: 1243-1249.
Caraceni, A., Zecca, E., Martini, C. & Conno, F. D. (1999) Gabapentin as an adjuvant to opioid analgesia for neuropathic cancer pain. J Pain & Symp Manag 17: 441-445.
Catterall, W. (2000) Structure and regulation of voltage-gated Ca2+ channels. Annu Rev Cell Dev Biol 16: 521-55.
Cesena, R. M. & Calcutt, N. A. (1999) Gabapentin prevents hyperalgesia during the formalin test in diabetic rats. Neurosci Lett 262: 101-104.
Chaplan, S. R., Pogrel, J. W. & Yaksh, T. L. (1994) Role of voltage-dependent calcium channel subtypes in experimental tactile allodynia. J Pharmacol Exp Ther 269 1117-1123.
Cheng, J-K., Pan, H-L. & Eisenach, J. C. (2000) Antiallodynic effect of intrathecal gabapentin and its interaction with clonidine in a rat model of postoperative pain.
Anesthesiology 92: 1126-1131.
Cribbs, L. L., Lee, J. H., Yang, J., Satin, J., Zhang, Y., Daud, A., Barclay, J., Williamson, M. P., Fo,x M., Rees, M. & Perez-Reyes, E. (1998) Cloning and characterization of alpha1H from human heart, a member of the T-type Ca2+ channel gene family. Circulation Research 83:103-109
De Waard, M., Gurnet, C. A. & Campbell, K. T. (1996) Structural and functional diversity of voltage activated calcium channels. In; Narahashi T. ed. Ion Channels. Plenum Press, NY. Di Trapani, G., Mei, D., Marra, C., Mazza, S. & Capuano, A. (2000) Gabapentin in the prophylaxis of migraine: a double-blind randomized placebo-controlled study. Clin Ter 151: 145-148.
Dooley, D. (1999) Lomerizine. Current Opinion in CPNS Investigational Drugs 1: 116-125.
Dubel, S. J., Starr, T. V., Hell, J., Ahlijanian, M. K., Enyeart, J. J., Catterall, W. A. & Snutch, T. P. (1992) Molecular cloning of the alpha-I subunit of an omega-conotoxin-sensitive calcium channel. Proc Natl Acad Sci USA 89: 5058-5062.
Dunlap, K., Luebke, J. I. & Turner, T. J. (1995) Exocytotic Ca2+ channels in mammalian central neurons. Trends Neurosci 18: 89-98.
Field, M. J., McCleary, S., Hughes, J. & Singh, L. (1999) Gabapentin and pregabalin, but not morphine and amitriptyline, block both static and dynamic components of mechanical allodynia induced by streptozocin in the rat. Pain 80: 391-398.
Fujita, Y., Mynlieff, M., Dirksen, R. T., Kim, M. S., Niidome, T., Nakai, J., Friedrich, T., Iwabe, N., Miyata, T. & Furuichi, T. (1993) Primary structure and functional expression of the omega-conotoxin-sensitive N-type calcium channel from rabbit brain. Neuron 10: 585-598.
Gomora, J. C., Daud, A. N., Weiergraber, M. & Perez-Reyes, E. (2001) Block of cloned human T-type calcium channels by succinimide antiepileptic drugs. Mol Pharmacol 60: 1121-1132.
Gould, R. J., Murphy, K. M., Reynolds, I. J. & Snyder, S. H. (1983) Antischizophrenic drugs of the diphenylbutylpiperidine type act as calcium channel antagonists. Proc Natl Acad Sci USA 80: 5122-5125
Grantham, C. J. Main, M. J & Cannell, M. B. (1994) Fluspirilene block of N-type calcium current in NGF-differentiated PC12 cells. Brit J Pharmacol 111: 483-488.
Hatakeyama, S., Wakamori, M., Ino, M., Miyamoto, N., Takahashi, E., Yoshinaga, T., Sawada, K., Imoto, K., Tanaka, I., Yoshizawa, T., Nishizawa, Y., Mori, Y., Niidome, T. & Shoji, S. (2001) Differential nociceptive responses in mice lacking the alpha(1B) subunit of N-type Ca(2+) channels. Neuroreport 12(11): 2423-2427.
Heading, C. (1999) Ziconotide. Curr Opin CPNS Investigational Drugs 1: 153-166.
Houtchens, M. K., Richert, J. R., Sami, A. & Rose, J. W. (1997) Open label gabapentin treatment for pain in multiple sclerosis. Multiple Sclerosis 3: 250-253.
Huguenard, J. R. (1996) Low-threshold calcium currents in central nervous system neurons. Annu Rev Physiol 58: 329-48.
Ino, M., Yoshinaga, T., Wakamori, M., Miyamoto, N., Takahashi, E., Sonoda, J., Kagaya, T., Oki, T., Nagasu, T., Nishizawa, Y., Tanaka, I., Imoto, K., Aizawa, S., Koch, S., Schwartz, A., Niidome, T., Sawada, K. & Mori, Y. (2001) Functional disorders of the sympathetic nervous system in mice lacking the alpha 1B subunit (Cav 2.2) of N-type calcium channels. Proc Natl Acad Sci USA 98(9): 5323-5328
Janis, R. J & Triggle, D. J. (1991) In Calcium Channels: Their Properties, Functions, Regulation and Clinical Relevance. CRC Press, London.
Jones, O. T. (2002) Ca2+ channels and epilepsy. Eur J Pharmacol 447: 211-225.
Kim, C., Jun, K., Lee, T., Kim, S. S., McEnery, M. W., Chin, H., Kim, H. L., Park, J. M., Kim, D. K., Jung, S. J., Kim, J. & Shin, H. (2001) Altered nociceptive response in mice deficient in the alpha(1B) subunit of the voltage-dependent calcium channel. Mol Cell Neurosci 18(2): 235-245.
Kim, D., Song, I. Keum, S., Lee, T., Jeong, M. J., Kim, S. S., McEnery, M. W. & Shin, H. S. (2001) Lack of the burst firing of thalamocortical relay neurons and resistance to absence seizures in mice lacking the α1G T-type Ca²⁺ channels. Neuron 31: 35-45.
King, V. F., Garcia, M. L., Shevell, J. L., Slaughter, R. S. & Kaczorowski, G. J. (1989) Substituted diphenylbutylpiperidines bind to a unique high affinity site on the L-type calcium channel. Evidence for a fourth site in the cardiac calcium entry blocker receptor complex. J Biol Chem 264: 5633-5641.
Laird, M. A. & Gidal, B. E. (2000) Use of Gabapentin in the treatment of neuropathic pain. Annal Pharmacotherap 34: 802-807.
Lee, J. H., Daud, A. N., Cribbs, L. L., Lacerda, A. E., Pereverzev, A., Klockner, U., Schneider, T. & Perez-Reyes, E. (1999) Cloning and expression of a novel member of the low voltage-activated T-type calcium channel family. Journal of Neuroscience 19: 1912-1921.
Magnus, L. (1999) Nonepileptic uses of Gabapentin. Epilepsia 40 (Suppl 6): S66-S72.
Malmberg, A. B. & Yaksh, T. L. (1994) Voltage-sensitive calcium channels in spinal nociceptive processing: blockade of N- and P-type channels inhibits formalin-induced nociception. J Neurosci 14: 4882-4890.
Mathur, V. S. (2000) Ziconotide: A new pharmacological class of drug for the management of pain. Seminars in Anesthesia, Perioperative medicine and Pain 19: 67-75.
McCleskey, E. W. & Schroeder, J. E. (1991) Functional properties of voltage dependent calcium channels. Cur. Topics Membr 39: 295-326.
McRory, J. E., Santi, C. M., Hamming, K. S., Mezeyova, J., Sutton, K. G., Baillie, D. L., Stea, A., & Snutch, T. P. (2001) Molecular and functional characterization of a family of rat brain T-type calcium channels. J Biol Chem 276: 3999-4011.
Mikami, A., Imoto, K., Tanabe, T., Niidome, T., Mori, Y., Takeshima, H., Narumiya, S. & Numa, S. (1989) Primary structure and functional expression of the cardiac dihydropyridine-sensitive calcium channel. Nature 340: 230-233.
Miller, R. J. (1987) Multiple calcium channels and neuronal function. Science 235: 46-52.
Mori, Y., Friedrich, T., Kim, M. S., Mikami, A., Nakai, J., Ruth, P., Bosse, E., Hofmann, F., Flockerzi, V. & Furuichi, T. (1991) Primary structure and functional expression from complementary DNA of a brain calcium channel. Nature 350: 398-402
Nicholson, B. (2000) Gabapentin use in neuropathic pain syndromes. Acta Neurol Scand 101: 359-371.
Penn R. D. & Paice J. A. (2000) Adverse effects associated with the intrathecal administration of ziconotide. Pain 85: 291-296.
Perez-Reyes, E. (2003) Molecular physiology of low-voltage-activated T-type calcium channels. Physiol Rev 83: 117-161.
Perez-Reyes, E., Cribbs, L. L., Daud, A., Lacerda, A. E., Barclay, J., Williamson, M. P., Fox, M., Rees, M. & Lee J. H. (1998) Molecular characterization of a neuronal low-voltage-activated T-type calcium channel. Nature 391: 896-900.
Ridgeway, B., Wallace, M. & Gerayli, A. (2000) Ziconotide for the treatment of severe spasticity after spinal cord injury. Pain 85: 287-289.
Rowbotham, M., Harden, N. N., Stacey, B., Podolnick, P. & Magnus-Miller, L. (1998) Gabapentin for the treatment of postherpetic neuralgia: a multicentre, double-blind cross-over study. JAMA 280: 1837-1842.
Saegusa, H., Kurhara, T., Zong, S., Minowa, O., Kazuno, A., Han, W., Matsuda, Y., Yamanaka, H., Osanai, M., Noda, T. & Tanabe, T. (2000) Altered pain responses in mice lacking α1E subunit of the voltage dependent Ca channel. Proc Natl Acad Sci USA 97: 6132-6137.
Salemme, F. R., Spurlino, J. & Bone, R. (1997) Serendipity meets precision: the integration of structure-based drug design and combinatorial chemistry for efficient drug discovery. Structure 5: 319-324
Santi, C. M., Cayabyab, F. S., Sutton, K. G., McRory, J. E., Mezeyova, J., Hamming, K. S., Parker, D., Stea, A., & Snutch, T. P. (2002) Differential inhibition of T-type calcium channels by neuroleptics. J Neurosci 22: 396-403.
Sather, W. A., Tanabe, T., Zhang, J. F., Mori, Y., Adams, M. E. & Tsien, R. W. (1993) Distinctive biophysical and pharmacological properties of class A (BI) calcium channel alpha I subunits. Neuron 11: 291-303.
Sluka, K. A. (1998) Blockade of N- and P/Q-type calcium channels reduces the secondary heat hyperalgesia induced by acute inflammation. J Pharmacol Exp Ther 287: 232-237.
Snutch, T. P., Tomlinson, W. J., Leonard, J. P. & Gilbert, M. M. (1991) Distinct calcium channels are generated by alternative splicing and are differentially expressed in the mammalian CNS. Neuron 7:45-57
Soong, T. W., Stea, A., Hodson, C. D., Dubel, S. J., Vincent, S. R. & Snutch, T. P. (1993) Structure and functional expression of a member of the low voltage-activated calcium channel family. Science 260: 1133-1136
Stea, A., Soong T. W. & Snutch, T. P. (1994) Voltage-gated calcium channels. In: Handbook on Ion Channels. RA North (ed). CRC Press, p. 113-151.
Stea, A., Tomlinson, W. J., Soong, T. W., Bourinet, E., Dubel, S. J., Vincent, S. R. & Snutch, T. P. (1994) Localization and functional properties of a rat brain alpha 1A calcium channel reflect similarities to neuronal Q- and P-type channels. Proc Natl Acad Sci USA 91: 10576-10580.
Su, H., Sochivko, D., Becker, A., Chen, J., Jiang, Y., Yaari, Y. & Beck, H. (2002) Upregulation of a T-type Ca²⁺ channel causes a long-lasting modification of neuronal firing mode after status epiletpicus. J Neurosci 22: 3645-3655.
Taylor, C. P., Gee, N. S., Su, T.-Z., Kocsis, J. D., Welty, D. F., Brown, J. P., Dooley, D. J., Boden, P. & Singh, L. (1998) A summary of mechanistic hypotheses of gabapentin pharmacology. Epilepsy Res 29: 233-249.
Tomlinson, W. J., Stea, A., Bourinet, E., Charnet, P., Nargeot, J. & Snutch, T. P. (1993) Functional properties of a neuronal class C L-type calcium channel. Neuropharmacology 32: 1117-1126
Tumilowicz, J. J., Nichols, W. W., Cholon, J. J. & Greene, A. E. (1970) Definition of a continuous human cell line derived from neuroblastoma. Cancer Res 30(8): 2110-2118.
Vanegas, H. & Schaible, H-G. (2000) Effects of antagonists to high-threshold calcium channels upon spinal mechanism of pain, hyperalgesia and allodynia. Pain 85: 9-18.
Wang, Y-X., Pettus, M., Philips, C., Gao, D., Bowersox, S. S. & Luther, R. R. (1998) Antinociceptive properties of a selective neuronal N-type calcium channel blocker, ziconotide. (SNX-111), in rat model of post-operative pain. Soc Neurosci Abstr 24: 1626.
Wentworth, P. Jr., & Janda, K. D. (1998) Generating and analyzing combinatorial chemistry libraries. Curr Opin Biotechnol 9: 109-115.
Williams, M. E., Brust, P. F., Feldman, D. H., Patthi, S., Simerson, S., Maroufi, A., McCue, A. F., Velicelebi, G., Ellis, S. B. & Harpold, M. M. (1992) Structure and functional expression of an omega-conotoxin-sensitive human N-type calcium channel. Science 257: 389-395.
Williams, M. E., Feldman, D. H., McCue, A. F., Brenner, R., Velicelebi, G., Ellis, S. B. & Harpold, M. M. (1992) Structure and functional expression of alpha 1, alpha 2, and beta subunits of a novel human neuronal calcium channel subtype. Neuron 8: 71-84.

1. A compound of the formula: 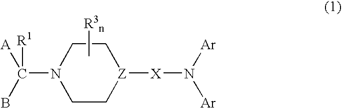
and salts or conjugates thereof,
wherein each of A and B is independently a 6-membered aromatic or nonaromatic, carbocyclic or heterocyclic moiety or is an aminoalkyl and wherein one and only one of A and B may be H or alkyl (1-8C);
R¹ is H or alkyl (1-8C);
Z is N or CHNR² wherein R² is H or alkyl (1-8C);
X is straight chain alkylene (1-4C) wherein a carbon adjacent to one nitrogen is in the form of C═O;
each R³ is independently a substituent selected from the group consisting of ═O, alkyl (1-8C), alkenyl (2-8C), alkynyl (2-8C), halo, CHF₂, CF₃, OCHF₂, OCF₃, CN, NO₂, NR₂, OR, SR, COR, COOR, CONR₂, NROCR, OOCR, SOR, SO₂R, SO₃R, SONR₂, SO₂NR₂, NRSOS, or NRSO₂R, wherein R is H or alkyl (1-8C), alkenyl (2-8C), alkynyl (2-8C), aryl and alkylaryl, and wherein two substituents on adjacent carbons may form an optionally substituted 5-7 membered ring;
n=0-2, and
Ar is a six-membered aromatic or heteroaromatic ring;
wherein each cyclic moiety included in a or b and each ar moiety in formula (1) may be substituted by one or more substituents selected from the group consisting of ═O (in nonaromatic cyclic moieties, alkyl (1-6C), halo, CHF₂, CF₃, OCHF₂, OCF₃, NO₂, NR₂, OR, SR, COR, COOR, CONR₂, NROCR, OOCR, SOR, SO₂R, SO₃R, SONR₂, SO₂NR₂, NRSOR, and NRSO₂R, wherein R is H or alkyl (1-8C), alkenyl (2-8C), alkynyl (2-8C), aryl or alkylaryl, and wherein two adjacent substituents may form a 5-7 membered ring, and
wherein any alkyl, cyclic or aryl group recited above may itself be substituted by ═O, halo, CHF₂, CF₃, OCHF₂, OCF₃, NO₂, NR₂, OR, SR, COR, COOR, CONR₂, NROCR, OOCR, SOR, SO₂R, SO₃R, SONR₂, SO₂NR₂, NRSOR, or NRSO₂R, wherein R is H or alkyl (1-8C), alkenyl (2-8C), alkynyl (2-8C), aryl or alkylaryl.

2. The compound of claim 1, wherein Z is N.

3. The compound of claim 1, wherein each Ar is independently phenyl or pyridinyl.

4. The compound of claim 1, R¹ is H.

5. The compound of claim 1, wherein each of A and B is independently substituted or unsubstituted phenyl or substituted or unsubstituted pyridyl.

6. The compound of claim 1, wherein the substituents on Ar, A and/or B are selected from the group consisting of CF₃, alkyl, halo, hydroxy and alkoxy.

7. The compound of claim 1, wherein n is 0 or each R³ is independently ═O or COOH.

8. The compound of claim 1, wherein X comprises one C═O group.

9. The compound of claim 8, wherein said C═O group is adjacent the nitrogen to which the Ar are attached.

10. The compound of claim 8, wherein said C═O group is adjacent Z.

11. The compound of claim 1, which is selected from the group consisting of
1-(4-benzhydryl-piperazin-1-yl)-2-diphenylamino-ethanone;
1-{4-[(2,4-dimethyl-phenyl)-phenyl-methyl]-piperazin-1-yl}-2-diphenylamino-ethanone;
1-{4-[(2,4-dichloro-phenyl)-phenyl-methyl]-piperazin-1-yl}-2-diphenylamino-ethanone;
1-{4-[(4-chloro-phenyl)phenyl-methyl]-piperazin-1-yl}-2-diphenylamino-ethanone;
1-{4-[(3-chloro-phenyl)-phenyl-methyl]-piperazin-1-yl}-2-diphenylamino-ethanone;
1-{4-[(2-chloro-phenyl)-phenyl-methyl]-piperazin-1-yl}-2-diphenylamino-ethanone;
1-{4-[(2,3-dichloro-phenyl)-phenyl-methyl]-piperazin-1-yl}-2-diphenylamino-ethanone;
1-[4-(benzo[1,3]dioxol-5-yl-phenyl-methyl)-piperazin-1-yl]-2-diphenylamino-ethanone;
2-diphenylamino-1-{4-[(4-methoxy-phenyl)-(4-trifluoromethyl-phenyl)-methyl]-piperazin-1-yl}-ethanone;
2-diphenylamino-1-[4-(1-methyl-piperidin-3-ylmethyl)-piperazin-1-yl]-ethanone;
2-diphenylamino-1-(4-pyridin-3-ylmethyl-piperazin-1-yl)-ethanone;
2-diphenylamino-1-(4-pyrid in-2-ylmethyl-piperazin-1-yl)-ethanone;
2-diphenylamino-1-[4-(phenyl-pyridin-3-yl-methyl)-piperazin-1-yl]-ethanone;
2-diphenylamino-1-[4-(phenyl-pyridin-2-yl-methyl)-piperazin-1-yl]-ethanone;
1-{4-[(4-tert-butyl-phenyl)-phenyl-methyl]-piperazin-1-yl}-2-diphenylamino-ethanone;
2-(4-benzhydryl-piperazin-1-yl)-N,N-diphenyl-acetamide;
2-{4-[(2,4-dichloro-phenyl)-phenyl-methyl]-piperazin-1-yl}-N,N-diphenyl-acetamide;
2-{4-[(2,4-dimethyl-phenyl)-phenyl-methyl]-piperazin-1-yl}-N,N-diphenyl-acetamide;
2-{4-[(4-chloro-phenyl)-phenyl-methyl]-piperazin-1-yl}-N,N-diphenyl-acetamide;
2-{4-[(3-chloro-phenyl)-phenyl-methyl]-piperazin-1-yl}-N,N-diphenyl-acetamide;
2-{4-[(2-chloro-phenyl)-phenyl-methyl]-piperazin-1-yl}-N,N-diphenyl-acetamide;
2-{4-[(2,3-dichloro-phenyl)-phenyl-methyl]-piperazin-1-yl}-N,N-diphenyl-acetamide;
2-[4-(benzo[1,3]dioxol-5-yl-phenyl-methyl)-piperazin-1-yl]-N,N-diphenyl-acetamide;
2-{4-[(4-methoxy-phenyl)(4-trifluoromethyl-phenyl)-methyl]-piperazin-1-yl}-N,N-diphenyl-acetamide;
2-[4-(1-methyl-piperidin-4-ylmethyl)-piperazin-1-yl]-N,N-diphenyl-acetamide;
2-[4-(3-dimethylamino-propyl)-piperazin-1-yl]-N,N-diphenyl-acetamide;
2-[4-(1-methyl-piperidin-3-ylmethyl)-piperazin-1-yl]-N,N-diphenyl-acetamide;
N,N-diphenyl-2-[4-(phenyl-pyridin-3-yl-methyl)-piperazin-1-yl]-acetamide;
N,N-diphenyl-2-[4-(phenyl-pyridin-2-yl-methyl)-piperazin-1-yl]-acetamide;
2-{4-[(4-tert-butyl-phenyl)-phenyl-methyl]-piperazin-1-yl}-N,N-diphenyl-acetamide;
2-{4-[(4-methoxy-phenyl)-phenyl-methyl]-piperazin-1-yl}-N,N-diphenyl-acetamide;
2-(4-benzhydryl-2,3-dioxo-piperazin-1-yl)-N,N-diphenyl-acetamide;
2-(4-benzhydryl-2,5-dioxo-piperazin-1-yl)-N,N-diphenyl-acetamide; and
1-benzhydryl-4-(2-diphenylamino-acetyl)-piperazine-2,5-dione.

12. The compound of claim 11, which is selected from the group consisting of
1-(4-benzhydryl-piperazin-1-yl)-2-diphenylamino-ethanone;
1-{4-[(2,4-dimethyl-phenyl)-phenyl-methyl]-piperazin-1-yl}-2-diphenylamino-ethanone;
1-{4-[(2,4-dichloro-phenyl)-phenyl-methyl]-piperazin-1-yl}-2-diphenylamino-ethanone;
-{4-[(4-chloro-phenyl)-phenyl-methyl]-piperazin-1-yl}-2-diphenylamino-ethanone;
1-{4-[(3-chloro-phenyl)-phenyl-methyl]-piperazin-1-yl}-2-diphenylamino-ethanone;
1-{4-[(2-chloro-phenyl)-phenyl-methyl]-piperazin-1-yl}-2-diphenylamino-ethanone;
1-{4-[(2,3-dichloro-phenyl)-phenyl-methyl]-piperazin-1-yl}-2-diphenylamino-ethanone;
2-diphenylamino-1-[4-(1-methyl-piperidin-3-ylmethyl)-piperazin-1-yl]-ethanone;
2-(4-benzhydryl-piperazin-1-yl)-N,N-diphenyl-acetamide;
2-{4-[(2,4-dichloro-phenyl)-phenyl-methyl]-piperazin-1-yl}-N,N-diphenyl-acetamide;
2-{4-[(2,4-dimethyl-phenyl)-phenyl-methyl]-piperazin-1-yl}-N,N-diphenyl-acetamide;
2-{4-[(4-chloro-phenyl)-phenyl-methyl]-piperazin-1-yl}-N,N-diphenyl-acetamide;
2-{4-[(3-chloro-phenyl)-phenyl-methyl]-piperazin-1-yl}-N,N-diphenyl-acetamide;
2-{4-[(2-chloro-phenyl)-phenyl-methyl]-piperazin-1-yl}-N,N-diphenyl-acetamide;
2-{4-[(2,3-dichloro-phenyl)-phenyl-methyl]-piperazin-1-yl}-N,N-diphenyl-acetamide;
2-[4-(1-methyl-piperidin-4-ylmethyl)-piperazin-1-yl]-N,N-diphenyl-acetamide; and
2-[4-(3-dimethylamino-propyl)-piperazin-1-yl]-N,N-diphenyl-acetamide.

13. The compound of claim 12, which is 1-{4-[(2,3-dichloro-phenyl)-phenyl-methyl]-piperazin-1-yl}-2-diphenylamino-ethanone.

14. A pharmaceutical composition which comprises the compound of claim 1 in admixture with a pharmaceutically acceptable excipient.

15. A method to treat a condition mediated by a calcium channel activity which method comprises administering to a subject in need of such treatment an amount of the compound of claim 1 sufficient to treat said condition.

16. The method of claim 15, wherein said condition is selected from the group consisting of stroke, anxiety, epilepsy, head trauma, migraine, inflammatory bowel disease, overactive bladder, irritable bowel syndrome, interstitial colitis and chronic pain, inflammatory pain, neuropathic pain, acute pain, schizophrenia, anxiety, depression, neural degenerative disorders, drug and alcohol addiction and withdrawal; cardiovascular conditions; sleep disorders, cancer, diabetes, male contraception and sexual dysfunction.

17. A method to treat a condition selected from the group consisting of stroke, anxiety, epilepsy, head trauma, migraine, inflammatory bowel disease, overactive bladder irritable bowel syndrome, interstitial colitis and chronic pain, inflammatory pain, neuropathic pain, acute pain, schizophrenia, anxiety, depression, neural degenerative disorders, drug and alcohol addiction and withdrawal; cardiovascular conditions; sleep disorders, cancer, diabetes, male contraception and sexual dysfunction which method comprises administering to a subject in need of such treatment an amount of the compound of claim 1 sufficient to treat said condition.
